(19) United States
(12) Patent Application Publication
(10) Pub. No.: US 2014/0303617 A1
(43) Pub. Date: Oct. 9, 2014

(54) INTRAVASCULAR NERVE ABLATION DEVICES & METHODS

(71) Applicant: Neuro Ablation, Inc., Minneapolis, MN (US)

(72) Inventor: Jin Shimada, Grantsburg, WI (US)

(73) Assignee: Neuro Ablation, Inc., Minneapolis, MN (US)

(21) Appl. No.: 14/197,727

(22) Filed: Mar. 5, 2014

Related U.S. Application Data
(60) Provisional application No. 61/772,844, filed on Mar. 5, 2013.

(51) Int. Cl. A61B 18/14

(52) U.S. Cl. CPC A61B 18/1492; A61B 2018/00434
USPC 606/41

(57) ABSTRACT

Methods, systems, and devices for ablating nerves within an artery. The method includes selecting an ablation device having an outer guiding catheter and an inner treatment catheter including an expandable element and an ablative element on the expandable element. The ablation device is selected such that the expandable element is sized to apply an outward force against a portion of the inner wall of the artery substantially sufficient to hold the artery open during an arterial spasm event. The method can include advancing the ablation device to the treatment location, positioning the treatment catheter out of the guiding catheter, expanding the expandable element such that after expansion the expandable element applies the outward force against the portion of the inner wall of the artery, and ablating the inner wall of the artery using the ablation element after expanding the expandable element and without moving the treatment catheter.

### CROSS REFERENCE TO RELATED APPLICATION

This application claims priority to U.S. Provisional Patent Application No. 61/772,844, filed Mar. 5, 2013, the entire contents of which are incorporated herein by reference.

### FIELD

This document relates to methods and devices for nerve monitoring and/or ablation, in particular for nerve monitoring and/or ablation within a vessel such as an artery.

### BACKGROUND

Hypertension is a common disease which can have serious adverse consequences, including an increased risk of stroke, damage to organs including the heart, kidneys, brain, blood vessels and retinas. However, while hypertension is serious and numerous medications exist which attempt to control hypertension, in many cases it remains difficult to manage. For many patients, medications only partially reduce blood pressure and the patients remain at risk.

The difficulty in controlling blood pressure may be due to the complex nature of blood pressure maintenance by the body. Blood pressure is affected by multiple interrelated factors including cardiac activity, the degree of vasoconstriction/vasodilation, the degree of sympathetic stimulation, kidney function, salt and water consumption and balance, the amount of renin/angiotensin produced by the kidneys, and the presence of any abnormalities of the sympathetic nervous system, as well as possibly other unknown factors.

The kidneys play a key role in blood pressure regulation. Sympathetic nerve stimulation to the kidneys results in the production of renin, retention of sodium and water, and changes in renal blood flow, all of which lead to increased blood pressure. Through a system of interactions with other organs, the production of renin ultimately leads to the production of aldosterone, which causes the conservation of sodium, the secretion of potassium, increased water retention and increased blood pressure. An interruption of the renin-angiotensin-aldosterone system is, therefore, one method of reducing hypertension. For example, therapeutic agents such as angiotensin converting enzyme (ACE) inhibitors, angiotensin receptor blockers (ARBs), and renin inhibitors reduce blood pressure by effecting this system. More recently, attempts have been made to reduce renin production and, therefore, reduce blood pressure by surgically transecting the sympathetic nerves to the kidneys to prevent sympathetic stimulation of the kidneys.

Recent studies have successfully reduced blood pressure in hypertensive patients through the use of ablation of the sympathetic nerves within the renal arteries. The ablation is performed through a catheter and radiofrequency (RF) energy is applied to the interior of the arteries in linear arcs that extend circumferentially around the artery. A single arc may extend around the entire artery or a series of arcs may be created. The arcs in the series of arcs may be spaced apart longitudinally somewhat but are overlapping radially such that the entire inner circumference is ablated by a line of ablation at some point along the length of the artery. In either case, the result is that the ablated arcs transect all nerves running through the walls of the renal arteries. By encircling the arteries with lines of ablation, the surgeon is sure to transect the renal nerves, even though the actual locations of the nerves are unknown.

Because renal artery ablation surgeries have only been performed relatively recently, the long term effectiveness and the risk of long term side effects from such surgeries is unknown. Due to the vital nature of the kidneys and the necessity of maintaining adequate blood flow to these organs, the risk that such surgeries could lead to scarring and stenosis of the renal arteries is an important consideration. If significant stenosis were to occur, the result could be a loss of kidney function, which could be more problematic than the initial hypertension. A more refined approach to renal nerve ablation is, therefore, desirable.

Similarly, chronic pain, heart failure, sleep apnea, diabetes (types 1 and 2), atrial fibrillation and/or diet to reduce or control obesity can be managed and/or treated through a variety of means, including numerous medications which attempt to control the diseases, however, in many cases they remain difficult to manage. Sympathetic nerve stimulation of some organs may contribute to the adverse effects and/or the progress of these conditions. It is thought that by surgically transecting the sympathetic nerves to certain organs, in order to reduce and/or prevent sympathetic stimulation, may aid in treatment.

Likewise, altering sympathetic nerve stimulation, by surgically transecting the sympathetic nerves, to the brain, to the stomach, to the esophagus and/or to alter other sympathetic nerve stimulation that is associated with a neurological response, may also have beneficial applications.

### SUMMARY

In one aspect, this disclosure can feature a method of ablating nerves within an artery of a patient at a treatment location. The method can include selecting an ablation device which may include an outer guiding catheter and an inner treatment catheter. The treatment catheter may include an expandable element and an ablative element located on the expandable element. The selection of the ablation device may be such that the expandable element is of a size to apply an outward force against a portion of an inner wall of the artery that is substantially sufficient to hold the artery open during an arterial spasm event. The method may further include advancing the ablation device to the treatment location, advancing the treatment catheter out of the guiding catheter, and expanding the expandable element. After expansion, the expandable element may apply the outward force against the portion of the inner wall of the artery. After expanding the expandable element the method may include ablating the inner wall of the artery using the ablation element without moving the treatment catheter.

Implementations of the method may include one or more of the following features. For example, the ablation device may be selected such that the expandable element abuts the inner wall of the artery around a circumference of the artery. The treatment catheter may include a plurality of ablative elements on an outer surface of the expandable element, which are positioned to abut the inner wall of the artery around the circumference of the artery.

Implementations of the method may include one or more of the following features. For example, the method may further include ablating with one or more first ablative elements and then ablating with one or more second ablative elements without moving the expandable treatment catheter. The method may further include emitting a stimulation pulse prior to ablation and detecting a physiological response and/or electrical response of stimulated nerve activity using the ablation device. The method may further include emitting a second stimulation pulse after ablation and detecting the physiological response and/or electrical response of nerve activity using the ablation device. The physiological response may include blood flow, blood pressure, or blood flow and blood pressure, for example, and the treatment location may be the renal artery.

In another aspect, this disclosure can feature an ablation device that may include a guiding catheter that has an inner lumen, and a treatment catheter within the guiding catheter. The treatment catheter may include a plurality of electrodes and an expandable element. The expandable element may have a first state, when located within the guidance catheter lumen, which may have a first diameter. The expandable element may have a second state, that is expanded relative to the first state, and may have a second diameter and an outer surface. The second diameter may be greater than the first diameter. The plurality of electrodes may be located on the outer surface of the expandable element. The expandable element may be expanded in an artery having a diameter which is less than the second diameter. The expandable element may be configured to apply a radially outward force against the artery, sufficient to hold the artery open during a spasm event. The expandable element may be configured to expand from the first state to the second state for ablation, and then to return to the first state to allow the treatment catheter to be retracted into the guiding catheter for removal of the ablation device.

Implementations of the device may include one or more of the following features. For example, the expandable element may be self-expanding, and may include a mesh. The expandable element may be cylindrical, may be a spring coil, may have parabolic shaped sidewalls, and/or may be basket shaped and include a plurality of arms which may be inflatable.

In some embodiments, a method of monitoring nerve activity of a patient within an artery includes selecting a monitoring device including an outer guiding catheter and an inner monitoring device, the monitoring device comprising an expandable element, an electrical stimulation element and an electrical detection element, wherein each of the electrical stimulation element and the electrical detection element are located on an outer surface of the expandable element, and wherein the expandable element is configured to apply an outward force against a portion of an inner wall of the artery substantially sufficient to hold the artery open during an arterial spasm event. The method further includes advancing the ablation device to the treatment location, advancing the monitoring device out of the guiding catheter, expanding the expandable element such that after expansion, the expandable element applies the outward force against the portion of the inner wall of the artery, emitting a first stimulation pulse from the stimulation element, and detecting nerve activity produced in response to the first stimulation pulse with the detection element. In some embodiments, the method further includes, after detecting nerve activity produced in response to the first stimulation pulse ablating the inner wall of the artery using the ablation element, after ablating emitting a second stimulation pulse from the stimulation element, and detecting the presence or absence of nerve activity produced in response to the second stimulation pulse with the detection element. The monitoring device may be selected such that the expandable element abuts the inner wall of the artery around a circumference of the artery. The method may be performed within the renal artery to monitor sympathetic nerve activity.

### BRIEF DESCRIPTION OF THE DRAWINGS

FIG. 1 is a partial cut-away side view of a catheter, which includes a guiding catheter and a therapeutic catheter, according to various embodiments.

FIG. 2 is a distal end view of a catheter, which includes a guiding catheter and a therapeutic catheter, according to some embodiments.

FIG. 3 is a partial cut-away side view of a catheter, which includes a guiding catheter and a therapeutic catheter that is extended distally from the guiding catheter, according to various embodiments.

FIG. 4 is a distal end view of a therapeutic catheter according to some embodiments.

FIG. 5 is a side view of a catheter, which includes a guiding catheter and a therapeutic catheter, within the lumen of a renal artery, according to various embodiments.

FIG. 6 is a side view of a catheter, which includes a guiding catheter and a therapeutic catheter that is extended distally from the guiding catheter, within the lumen of a renal artery, according to various embodiments.

FIG. 7 is a distal end view of a therapeutic catheter according to some embodiments.

FIG. 8 is an elevation side view of a portion of a therapeutic catheter according to some embodiments.

FIG. 9 is an elevation side view of a portion of a therapeutic catheter according to some embodiments.

FIG. 10 is a distal end view of a therapeutic catheter according to some embodiments.

FIG. 11 is an elevation side view showing a detail portion of a therapeutic catheter according to some embodiments.

FIG. 12 is an elevation side view of a portion of a therapeutic catheter according to some embodiments.

FIG. 13 is a distal end view of a therapeutic catheter according to some embodiments.

FIG. 14 is an elevation side view of a portion of a therapeutic catheter according to some embodiments.

FIG. 15 shows a catheter introduced through the arterial system of a patient into the lumen of the renal artery, and a controller, according to some embodiments.

FIG. 16 is a schematic diagram of a system for nerve mapping and ablation according to some embodiments.

FIG. 17 shows a system for nerve mapping and ablation according to some embodiments.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, dimensions, and manufacturing processes are provided for selected elements, and all other elements employ that which is known to those of ordinary skill in the field of the invention. Those skilled in the art will recognize that many of the noted examples have a variety of suitable alternatives. In order to describe the methods and devices with clarity and precision, specific reference may be made to renal nerve ablation, epicardial ablation and/or other specific procedures. It will be appreciated, however, that embodiments of the devices and methods disclosed herein can be applicable to treat or alter other nerve functions, including other sympathetic nerve functions and additionally may be used in other ablation applications. Accordingly, references to particular procedures or target sites for ablation should not be read to limit applicability, but rather as clarifying examples.

The methods and devices described herein may be embodied in various forms, and may be used for nerve localization, ablation and/or for monitoring of the progress of nerve ablation in various body locations. For example, selective nerve ablation may be used for the treatment and/or management of chronic pain, heart failure, sleep apnea, diabetes (types 1 and 2), atrial fibrillation and/or diet to reduce or control obesity. The methods and devices described herein may also be used in the context of altering nerve stimulation to the brain, to the stomach, to the esophagus, and/or to alter any other nerve stimulation that is associated with a neurological response. In addition, the methods and devices described herein can also be used in epicardial ablation, carpal tunnel ablation, or in other locations where ablation may be desired. The devices used for nerve localization, monitoring and/or ablation may be configured to include an expandable member which carries the ablative elements such as the electrodes. After deployment, the expendable element may expand, such as by self-expansion, to bring the ablative elements, such as the electrodes, fully into contact with the inner wall of the vessel. Furthermore, the expandable elements may exert a radially directed outward force against the vessel wall to counteract any vessel contraction or spasm that could occur and which might otherwise complicate or thwart the ablation procedure.

For hypertension treatment, embodiments of the invention may selectively localize and ablate the branches of the renal nerves within the renal arteries. By identifying treatment locations such as by mapping the location of the branches of the renal nerves, the ablation can be performed through the tissue of the renal artery that overlies the nerve branch and can be monitored to ensure success. The amount of ablation delivered to the tissue of the walls of the renal artery can, therefore, be reduced, and the risk of side effects can also be reduced. In addition, by knowing effective treatment locations such as the locations of the branches of the renal nerves, the clinician can decide whether to ablate all or only some of the branches of the renal nerves. If less than all of the branches of the nerve are ablated, some sympathetic stimulation of the kidney can be maintained, which may have some clinical benefits. Furthermore, in some embodiments, the ablation may be performed using a non-electric modality such as laser, cryoablation, high frequency ultrasound, thermo ablation, or ablation by microwave energy, for example. In some such embodiments, the progress of nerve ablation can be monitored during ablation, such as by monitoring changes in electrical conduction of the ablated nerve resulting in changes in stimulated electrical activity of the nerve and/or changes in physiological parameters. The clinician can then determine when to stop ablation, which may be when the nerve branch or branches are completely ablated or may be after only partial ablation of the nerve branch or branches.

For example, a branching system of nerves known as the renal plexus provides sympathetic stimulation to the kidneys. These nerves of the renal plexus extend to the kidneys by traveling within the walls of the renal arteries. The nerves divide into multiple branches as they extend distally within the walls of the renal arteries. Embodiments of the invention reduce or eliminate sympathetic stimulation to the kidneys by ablating some or all of these nerves. In some embodiments, the locations of one or more of all of the branches of these nerves are specifically identified within the walls of the renal arteries and ablation is then performed at these locations. As opposed to random or nonspecific ablation patterns, monitoring of the sympathetic nerve activity with or without electrical nerve stimulation and specific identification or mapping of the nerves reduces the amount of renal artery tissue which is ablated such that only the tissue at or around the nerve branches is ablated. This, in turn, may reduce the risk for long term complications such as renal artery stenosis and kidney failure. Furthermore, specific mapping of each of the nerve branches allows a clinician to decide whether to ablate all or less than all of the branches. For example, it may be desirable to retain some amount of nerve stimulation and, therefore, a clinician may decide to ablate only a portion of the branches while leaving other branches intact without ablating them.

Alternatively, ablation may be performed in other locations such as within other vessels. Examples of other locations include any vessel having nerves within the vessel wall which may be ablated from the lumen of the vessel.

When a catheter, or catheter-based device, such as an ablation device, is inserted into and maneuvered within a vessel such as an artery, the artery may contract or spasm. Such a spasm may interfere with the procedure, by preventing advancement or movement of the catheter in the artery. Various embodiments therefore, include one or more expandable member on which one or more ablation elements are located. The expandable member may be located on, or may be an integral part of a delivery device, such as a therapeutic catheter, that may be delivered to an intravascular location within a guiding catheter. The expandable member may expand after it has passed through the distal end of the guiding catheter. The expandable member may then expand automatically, by self-expansion, when no longer constrained by the guiding catheter. In other embodiments, the expandable member may be expanded by active deployment by the clinician. For example, the expandable member may be expanded by a clinician by pulling and/or pushing wires attached to the expandable member to expand the mesh from the catheter handle.

After the expandable member is deployed, the radially outermost surface of the expandable member may fully contact the inner wall of the vessel. Depending upon the shape of the expandable member, this contact may be continuous around the entire circumference of the vessel wall. In some embodiments, the contact areas may be discontinuous but may nevertheless occur around the circumference of the vessel wall in a manner sufficient to hold the vessel open in response to spasm.

Because the ablation elements are in contact with the inner vessel wall after expansion of the expandable element, once the device is expanded in a desired position, ablation can be performed at the position even if vessel spasm occurs. In particular, in embodiments including multiple ablation elements, many or all of the ablation elements may be in contact with the vessel wall after deployment and expansion of the expendable member. Individual ablation elements can, therefore, be selected by a clinician and used for mapping, stimulation, and/or ablation, with the option to use multiple selected ablation elements at the same time or consecutively, without the need to reposition the device. In this way, once the expandable member is deployed, the entire procedure can be performed without repositioning the device, so that the entire ablation can be performed even if spasm occurs. In addition, the ablation elements selected by the clinician may be determined based on the mapping and/or stimulation performed previously at that location after expansion of the expandable member without moving the device.

Embodiments of the invention may identify treatment locations such as the location of one or more nerves or nerve branches such as the sympathetic nerves or nerve branches as they travel within the walls of arteries prior to performing ablation. In some embodiments, the treatment location such as the locations of the nerve branches are identified by delivery of an electrical pulse at a first location and detection of the electrical pulse at a second location. The first and second locations may be endoluminal, within the arterial wall. In some embodiments, both the first and second locations are within an artery. The first location may be proximal (closer to the aorta) while the second location may be distal (closer to the organ). In other embodiments, the first location is distal while the second location is proximal.

In some embodiments, the treatment location identification, such as nerve mapping, is performed using a catheter based electrode device. The catheter may include an electrical stimulation element which may be a first electrode for delivery of the stimulation electrical pulse at a first location and an electrical detection element which may be a second electrode for detection of the stimulated pulse of nerve activity at the second location. In embodiments in which the first pulse is delivered at a proximal location, the first electrode may be located proximally on the catheter and the second electrode may be located distally on or at the distal tip of the catheter. In embodiments in which the first pulse is delivered at a distal location, the first electrode may be located distally on the catheter or at the distal tip and the second electrode may be located proximally. In this way, a location for treatment may be identified. The same process of stimulation and detection of the electrical pulse may be used to monitor nerve activity. This may be performed for diagnostic purposes, to evaluate the condition of the nerve, either as part of an ablation procedure (before, during, and/or after ablation) or separately from an ablation procedure. For example, it may be done as part of an evaluation for planning an ablation procedure, or may be done as a follow up after an ablation procedure, such as by detecting the nerve activity some time after completion of an ablation procedure, with or without comparing the nerve activity to a baseline nerve conduction prior to the ablation procedure.

In some embodiments, identification of treatment locations and monitoring of the nerve activity like that described above may be done without nerve stimulation. In such embodiments, the catheter based electrode device may include an electrical detection element such as an electrode, which can detect spontaneous nerve activity through the vessel wall. In such embodiments, the electrical signal produced by inherent nerve activity may be detected, without the use of a stimulation pulse. Such detection may be used as part of an ablation procedure, to identify a treatment location and to monitor and confirm the success of the procedure as indicated by a change in the detected inherent nerve activity resulting from the ablation (such as a loss of nerve activity), such as by monitoring before, during, and/or after the ablation procedure. The monitoring of nerve activity may also be separate from an ablation procedure, such as to evaluate and diagnose the condition of the nerves, or may be performed as a follow up to an ablation procedure, with or without comparison to a baseline.

The catheter may be positioned such that the first and second electrodes abut or are in close proximity to the inner surface of an artery prior to delivery of the electrical pulse, such as by expansion of the expandable member. In some embodiments, the catheter may be designed to apply an outward radial force to the inner walls of the artery, such that the artery can be held open and first and second electrodes are pressed against the inner surface of the artery. Once so positioned, the electrical pulse may be delivered. If the second electrode detects conduction of the pulse consistent with conduction by a nerve, then it is known that a nerve branch or branches located at or near the location of each electrode and the location may be used for monitoring and/or ablation. However, if conduction by a nerve is not detected or is not adequately detected, then the catheter may be repositioned such that the location of one or both of the electrodes is adjusted. In some embodiments, a catheter may have a sufficient plurality of electrodes, that a new selection of electrodes is possible, without moving the catheter within the artery. This process may be repeated until the delivery of a pulse through a nerve is detected. When nerve conduction is detected, the location of the nerve branch or branches within the wall of the renal artery is identified as being directly or nearly directly beneath each of the first and second electrodes. As such, these electrodes perform a mapping function and may be described as mapping electrodes. Alternatively, these electrodes may be used for monitoring the progress or result of ablation and may be described as monitoring electrodes.

Once the nerve location is identified, ablation may then be performed at either the first or second location or both. In some embodiments, ablation is performed using the same device as was used for mapping of the nerves. In some such embodiments, the ablation can be performed without moving the catheter. In other such embodiments, the catheter may be repositioned, such as by rotation and/or advancing or retracting the catheter to align the first and/or second locations with the ablation delivery mechanism. In some embodiments, the catheter is not repositioned at all or is only repositioned slightly for ablation, such that the mapping or monitoring electrodes are still able to be used for detecting nerve conduction during the ablation procedure.

In some embodiments, the catheter used for nerve mapping may include more than two electrodes, such as three, four or more electrodes. In such embodiments, a first electrode may deliver an electrical pulse to the artery luminal surface and the second, third, and if present, additional electrodes may monitor second, third, and additional locations on the luminal surface of the artery for conduction of the pulse by a nerve. In this way, multiple locations may be monitored for each pulse delivery.

Because of the small size of nerve branches within an artery, it may be preferable to deliver nerve pulses having small amplitudes for nerve mapping. For example, the energy pulse may have an amplitude of between about 0.1 mA and about 50 mA. The pulse rate may be between about 5 Hz and about 100 Hz. The pulse width may be between about 0.1 microseconds and about 100 microseconds. In some embodiments, a square pulse may be delivered, to be most clearly defined. In other embodiments, the pulse may be peaked or sinusoidal. The controller may detect conduction by a nerve branch or branches by the characteristics of the detected signal and may use resistance to screen out background noise.

As described herein, RF energy may be delivered to ablate the nerve branches identified by identification of monitoring or treatment locations such as by mapping, in accordance with some embodiments. Devices which may be used for the ablation using RF energy may be unipolar or bipolar, such as the ablation devices that are described elsewhere herein. Such devices may include electrodes for detection and/or monitoring of electrical conduction as described herein. According to the ablation treatment location, adjustment of the ablation energy may be required. As a result, devices may be used with an RF generator that is adjustable to achieve a desired wattage, such as about 2 Watts, though more or less may alternatively be used.

In some embodiments, ablation is performed at a series of locations, with sufficient energy to partially or completely ablate the nerve branch or branches as desired, such as RF energy applied for about 2 minutes at about 8 Watts. The first location may be distally located within an artery, and each subsequent treatment location may be more proximally located than the previous location. The device may be held stationary while new locations are identified after mapping or after a first ablation. In some embodiments, a stationary device can be used to identify and/or monitor all potential ablation locations prior to performing a first ablation. In some embodiments a stationary device can be used to alternately map ablation locations, and ablate some of those locations. For example, after a first mapping, three locations may be ablated. A second mapping may then be performed, which may be followed by the ablation of two additional locations. In addition, there may be a cooling period, such as a period of about 5 minutes, between ablations. A series of about 4 to about 6 ablations may be performed in some embodiments.

In other embodiments, the ablative energy is laser energy. In some such embodiments, the ablation device can include a YAG laser and a flexible power generator in order to accommodate treatment in a variety of locations.

Other forms of ablation may alternatively be used, including cryoablation, high frequency ultrasound ablation (HIFU), microwave, thermoablation (heat) or other types of ablation as may be invented in the future, using any of the catheter configurations disclosed elsewhere herein, or variations thereof. In each case, the ablation may be performed at a precise location through a wall of the artery to selectively ablate a nerve branch as described herein.

In some embodiments, the progress of the ablation may be monitored by the system before, after, and/or while the ablation is being performed, such as by detection of stimulated nerve conduction and nerve activity or physiological response. In some such embodiments, the progress of the ablation may be continuously monitored as ablative energy is delivered. In other embodiments, the progress of the ablation may be intermittently monitored as ablative energy is delivered. In still other embodiments, the delivery of ablative energy may be momentarily halted to detect the progress of the ablation. For example, the delivery of ablative energy may be momentarily halted at periodic intervals at which time the progress of the ablation may be detected.

In some embodiments, the progress of ablation is detected by the delivery of a pulse of energy. The energy pulse may be delivered and detected using the same first and second (or third or additional) electrodes as were used to deliver the energy pulse for localization of the nerve branch or branches that are being ablated. A single pulse of energy may be delivered or multiple pulses may be delivered at periodic intervals. For example, a series of pulses of energy may be delivered on a periodic basis as ablation is being performed. For example, pulses of energy may be delivered between about every 0.1 second and about every 5 seconds. In other embodiments, the pulses of energy may be delivered between about every 1 second and about every 5 seconds.

In some embodiments, the energy pulses delivered during monitoring may be identical to the pulses delivered during localization in that they have the same frequency, amplitude, and duration, and may also be delivered identically throughout monitoring. In this way, changes in the characteristic of the energy pulse may be detected as ablation proceeds. Such changes may then be interpreted to correspond to the effectiveness or amount of ablation of the nerve branch or branches. For example, the changes in the electrical pulse that may correspond to the effectiveness of the ablation may be a decreased amplitude and/or a time delay (a shifting of the position of the waveform) from the original baseline amplitude and conduction time. Other changes which may be detected include tissue resistivity changes and delays in the signal responses at the receiving electrode due to a change in the pathway as the resistance of the nerve increases and the signal follows a new lower resistance pathway, such as through a different nerve bundle.

In some embodiments, the progress of ablation may be monitored by monitoring physiological parameters, such as physiological parameters in the renal artery, kidney or elsewhere in the patient's body. For example, for renal nerve ablation, such parameters may include one or more of renal arterial blood pressure, renal arterial blood flow, renal artery diameter, renal vascular resistance, urine production rate, urinary sodium excretion rate, urinary potassium excretion rate, renin production, and/or renin excretion rate. Other physiological parameters that respond to partial and/or complete loss of sympathetic nerve stimulation of the kidneys or other locations may be used. For ablation in other locations, such parameters can be those that may be associated with the location of ablation treatment and/or those that may be associated with the condition being treated.

In some embodiments, the physiological parameter may be measured within the artery of interest, such as by using the same catheter as was used for nerve localization and/or ablation. Alternatively, a separate catheter or separate measuring method may be used. In embodiments in which measurements are made in the urine, a urinary catheter may be used in the bladder, for example. In embodiments in which heart rate is measured, an EKG or other cardiac monitor may be used, for example. In some embodiments, artery blood pressure, blood flow, diameter, and/or vascular resistance may be measured within the artery of interest using one or more sensors, such as a pressure sensor, flow sensor, ultrasonic sensor, or other known sensor technologies, which may or may not be a component of the mapping and/or ablation catheter.

Measurements of the physiological parameter may be made in order to determine the effectiveness of the ablation (amount of denervation). For example, a baseline measurement of the physiological parameter may be taken prior to ablation. Ablation may then be performed. The physiological measurement may then be repeated, possibly after waiting a certain time period after the ablation. The amount of change in the physiological parameter (if any) may then be used to determine whether the desired degree of denervation has been achieved and whether or not an additional course of ablative energy should be delivered to the nerve. If additional ablation is performed, the physiological parameter can be measured again and the process may be repeated until the change in the physiological parameter indicates that the desired amount of denervation has been achieved.

Alternatively, the effect of the nerve stimulation upon the physiological parameter may be used to assess the effectiveness of the ablation. In such embodiments, a baseline measurement of the physiological parameter may be obtained with and without stimulation of a nerve prior to ablation. Ablation of the nerve may then be performed. The physiological measurements may then be repeated, possibly after a time delay, both with and without stimulation of the ablated nerve. The amount of change in the physiological parameter due to nerve stimulation may change (decrease) as nerve conduction is decreased and may be finally eliminated by ablation. One or more ablation steps may, therefore, be performed, followed by measurements of the physiological parameter with and without stimulation of the ablated nerve, until the desired amount of denervation has been achieved. That the desired amount of denervation has been achieved, may be determined by observing that the amount of change of the physiological parameter caused by stimulation has changed (typically decreased), by a desired amount.

In each of the above examples regarding changes in physiological parameters, the amount of change in a physiological parameter, or the amount of change in the effect of stimulation on a physiological parameter, may be experimentally correlated to the amount of denervation (effectiveness of ablation). For example, the parameters and/or the changes in the parameters due to stimulation, may be correlated to the amount of denervation as determined by a series of measurements of the parameters, and/or changes in the parameters due to stimulation, in a test group of individuals and analyzing the data before and after ablation treatment. This data may then be used to correlate the measurements of physiological parameters in patients undergoing ablation to the amount of denervation achieved.

In some embodiments, the physiological parameter may be measured before and after ablation. The measurement after ablation may be immediately after cessation of ablation or may be after a delay period. In some embodiments, the physiological parameter may be measured during ablation as a way to monitor the progress of the ablation. In some embodiments, the physiological parameter may be measured before, during and after ablation. The measurement of the physiological parameter may supplement the measurement of the nerve conduction as an additional way of monitoring the effectiveness of the ablation. If both nerve conduction and one or more physiological parameter are used to assess the progress of ablation, they may both be performed simultaneously, or they may be performed separately.

The effectiveness of ablation treatment may thus be monitored using the pharmaceutical agent, according to some embodiments. For example, sympathetic nerves may be stimulated using a pharmaceutical agent that can be delivered to the renal artery by the mapping and/or ablation catheter. Similar to a method described above, the effectiveness of the ablation may be monitored by the amount of change in the effect of stimulation due to the pharmaceutical agent on a physiological parameter. One example of an appropriate pharmaceutical is norepinephrine, but other agents may alternatively be used. As in the method described above, the change in the effect of stimulation due to the pharmaceutical agent can be experimentally correlated in a test group of individuals before and after ablation. This data may then be used to correlate the change in measurements of physiological parameters due to the agent, in patients undergoing ablation, to the amount of denervation achieved.

In some embodiments, the clinician performing the ablation may elect to only partially ablate one or more of the nerve branches. In such embodiments, the clinician may elect to deliver ablative energy until ablation is partially complete as determined by monitoring the ablation progress as described herein. For example, the clinician may decide to ablate a nerve branch or branches by a certain amount, and this amount may be determined by the amount of decrease in the measured amplitude of the detected signal/nerve activity, by the amount of time delay in transmission of the detected signal/nerve activity, by the change in a physiological parameter, and/or by the change in the effect of nerve stimulation on the physiological parameter.

The clinician may then deliver the ablative energy to the nerve while continuously or intermittently monitoring the progress of the ablation. When the monitoring shows that the desired amount of ablation has been achieved, the clinician may stop delivery of the ablative energy.

In some embodiments, the clinician may desire to completely ablate the nerve or nerve branch but may still monitor the progress of nerve ablation during the delivery of ablative energy. The clinician may continue delivery of the ablation energy until it is determined that no nerve conduction is occurring, such as by an absence of detectable nerve delivery of the energy pulse, or by the measurement of the physiological parameter. The clinician may then discontinue delivery of the ablative energy. In some embodiments, the clinician may continue delivery of some additional amount of energy after complete ablation is detected to provide a margin of error or to allow for some nerve recovery in the future. However, in either case, by monitoring the progress of the ablation, the clinician can determine when to stop ablation (whether immediately or after a certain amount of time after completion of ablation). As such, by monitoring completeness of ablation, the clinician can be assured that the ablation procedure was successful, since the monitoring shows that nerve conduction is no longer occurring.

In addition to assuring that ablation has occurred as planned, the use of monitoring during the ablation procedure can allow the clinician to use less ablative energy even when complete ablation is desired. For example, the amount of energy needed to ablate a nerve or nerve branch within an artery such as the renal artery may vary among individuals, and could depend upon factors such as the size, age, gender, health status, or unique anatomy of the individual. In addition, even for an individual, the amount of energy needed to ablate a nerve branch may vary among the branches, depending, for example, upon the size of the branch or the depth of the branch within the tissue or the artery wall. Therefore, if the progress of the ablation is not monitored, a clinician would need to deliver the maximum amount of energy which could possibly be necessary to every nerve branch and every individual to assure complete ablation of each nerve branch. However, by monitoring the progress of the ablation, only the necessary amount of ablation needs to be delivered for each particular nerve branch because the effectiveness of the ablation can be observed. In this way, by monitoring the progress of the ablation, the delivery of unnecessary amounts of ablative energy beyond what is needed for ablation can be avoided. It is anticipated that by reducing the amount of ablative energy delivered to the tissue, less damage is caused to the artery wall and, therefore, the risk of complications such as stenosis are further reduced.

In some embodiments, the mapping and ablation device can include one or more temperature sensors to detect the temperature of the tissue at or near the ablation site. In some embodiments, the temperature sensors may be electrodes such as metal electrodes which may include MEMS technology to convert the temperature to an output voltage. In some embodiments, the temperature may be measured using fiber optics, by sending and receiving laser energy to detect changes in the radiance of the tissue relating to temperature, such as through non-touch thermal sensors. The temperature of the target tissue may be monitored during the ablation process to prevent damage to the tissue or to the blood, such as blood coagulation. For example, ablation may be stopped or decreased if the temperature of the tissue reaches about 72° F. or about 75° F., for example.

The mapping and ablation catheter device may include a steerable guide catheter portion for navigating the catheter to the appropriate location, and an ablation catheter portion which may include the ablation elements and may optionally include the mapping elements. The ablation catheter may reside within a lumen of the guide catheter and may include an ablation head that extends beyond the distal tip of the steerable guide catheter. The ablation head may include the mapping and/or monitoring and ablation elements and may also include ports for cooling solution entry and exit and temperature sensors. The ablation catheter may have an overall small diameter, such as about 6 French or less, making is easier to manipulate and position and making the use of the catheter less invasive. In some embodiments, the overall diameter of portions of the ablation catheter can expand from an initial small diameter to a diameter that is substantially larger, as is explained in detail elsewhere herein. In some embodiments, a small initial size for the catheter prior to expansion may be achieved through the use of conductive optical fibers and reduced numbers of conductive wires or the elimination of conductive wires, such as in laser ablation systems.

In some embodiments, the steerable guide catheter may include a compound curvature. In some embodiments, only the distal tip of the catheter may be movable, and the distal tip may be able to move in multiple dimensional axis, allowing the catheter to be steered to the target site even in embodiments in which the body of the catheter is not steerable.

During some intravascular ablation procedures, arterial spasming has been observed. Arterial spasming can result from the reaction of the arterial wall tissue to the presence of a foreign object (for example, the device itself), the delivery of a mapping pulse, and/or due to the delivery of an ablation pulse. In order to reach multiple target areas, some devices are designed to be rotated and/or axially moved during an ablation procedure. Such movements can trigger an arterial spasm and/or increase the likelihood that an arterial spasm will be triggered when a pulse is delivered. Arterial spasming can cause the artery to constrict, which can result in an increase in blood pressure and/or cause difficulty in rotating, advancing and/or retracting therapeutic devices that have been introduced into the arterial lumen. In addition, arterial spasms could also result in the movement of a correctly placed device, which could then result in a failure to reach all desired ablation sites or in ablation of an incorrect location.

As described elsewhere herein, methods for ablating and optionally for mapping are described that include the application of a radial force. Such a radial force can be applied to the inside wall of an artery, and the force can be of sufficient magnitude to hold open the artery during an arterial spasm. Such methods can include the use of, for example, one or more of the ablation devices that are described in the following paragraphs.

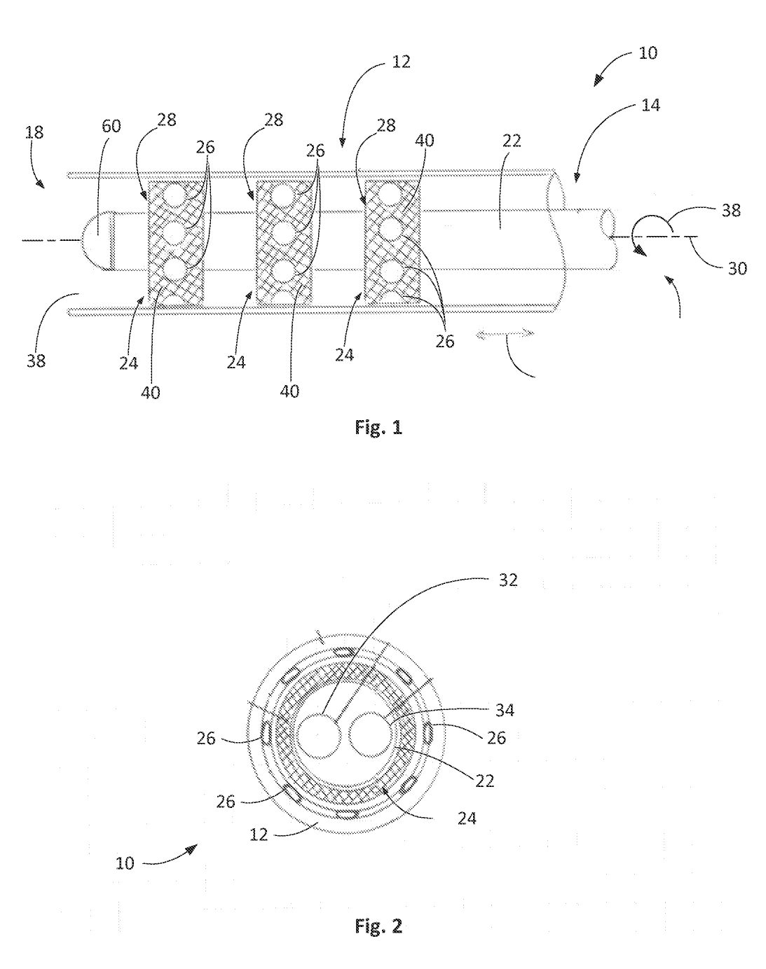

FIG. 1 shows a catheter 10, which can include a guiding catheter 12 and a therapeutic catheter 14, according to some embodiments. The therapeutic catheter 14 can be sized in order to fit within the lumen 16 of the guiding catheter 12. The guiding catheter 12 can have a distal end 18 and a proximal end. The therapeutic catheter 14 can have a distal end 20 and a proximal end. The therapeutic catheter 14 may comprise a catheter portion 22 and one or more expandable member 24 positioned along the length of the catheter portion 22 and on which one or more electrodes 26 may be located. In this figure, the expandable member 24 are in a contracted state within the guiding catheter 12.

In some embodiments, the expandable member 24 may be a generally cylindrical expandable element 28, such that the central axis of the cylindrical expandable element 28 may overlie the central axis 30 of the therapeutic catheter 14. The one or more electrodes 26 can be positioned on or near the outermost surface of the expandable member 24 and may be suitable for ablation and/or for stimulation and/or sensing (mapping). In alternative embodiments, the electrodes 24 may be replaced by alternative ablation elements such as cryogenic, laser, microwave, or other ablative elements.

FIG. 2 is a distal end view of the catheter 10 of FIG. 1. The therapeutic catheter 14 can optionally include sensor 60, such as a blood velocity sensor and/or a blood pressure sensor. The sensor 60 may be connected to a controller by a blood velocity sensor conductor 32 and/or blood pressure sensor conductor 34, which may be located within the lumen of the therapeutic catheter 14 or elsewhere. Other conductors may be used for other types of sensors. The expandable member 24 can be seen within the guiding catheter 12 and supporting the electrodes 26 located circumferentially around the outer periphery of the expandable member 24.

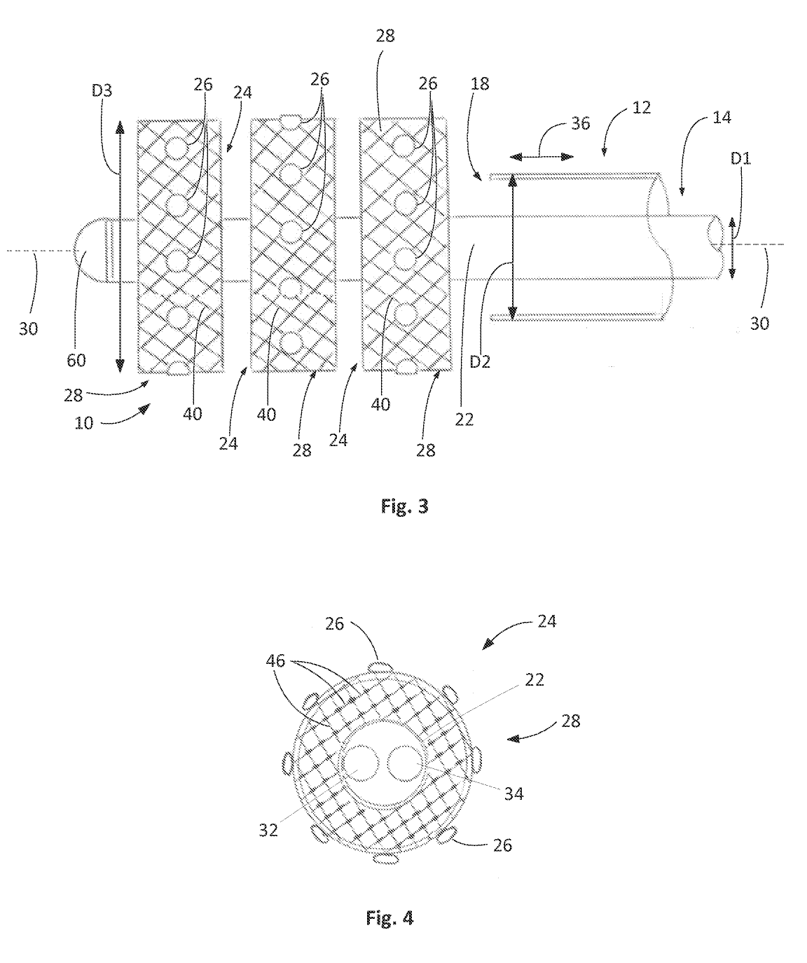

FIGS. 3 and 4 depict the catheter 10 of FIGS. 1 and 2 with the therapeutic catheter 14 extended beyond the distal end 18 of the guiding catheter 12. A side view is shown in FIG. 3 while an end view is shown in FIG. 4. The expandable member 24 have expanded in a radial direction, outwards from the catheter's central axis 30. In this expanded form, the expandable member 24 may have an expanded drum shape. The therapeutic catheter 14 can have an outer diameter that is D1 and the guiding catheter 12 can have an inner lumen diameter that is D2. In the contracted state, the expandable member 24 may have a diameter that is equal to D1 and/or is less than or approximately equal to D2 as the expandable member may abut the surface of the inner lumen of the guiding catheter 12. In the expanded state, the expandable member 24 may have an increased outer diameter D3. The size of D3 may depend upon the location in which the catheter 14 is used, and the appropriately sized catheter 14 may be selected accordingly. For example, when used in particular arteries such as the renal arteries, the catheter 14 may include an expandable member 24 sized to expand sufficiently to abut the wall of the particular artery and apply an outward radial force to the artery wall sufficient to prevent contraction of the artery in response to arterial spasm. For example, the size of each of D1, D2, and D3 may be between about 0.0080 and about 0.500, but with D1 less than D2 and D2 less than D3.

Referring now to FIGS. 1-4, in some embodiments the therapeutic catheter 14 can slide longitudinally as indicated by arrow 36 within the lumen 38 of the guiding catheter 202, such that the therapeutic catheter 14 can move relative to the guiding catheter 12 in a telescopic motion, to extend distally out and away from, as well as retract proximally into, the guiding catheter 12. The therapeutic catheter 14 can rotate about its own axis 224 as indicated by arrow 38 both within the lumen 38 of the guiding catheter 12, as well as when extended out from the guiding catheter 12.

In some embodiments, the therapeutic catheter 14 can be simultaneously moved in a rotational direction as well as in a longitudinal direction, such that movement in each direction can be independently controlled. In some embodiments, the therapeutic catheter 14 can only be moved in only one direction (rotational or longitudinal) at a time. In some embodiments, the therapeutic catheter 14 can be rotated both clockwise, and counter-clockwise.

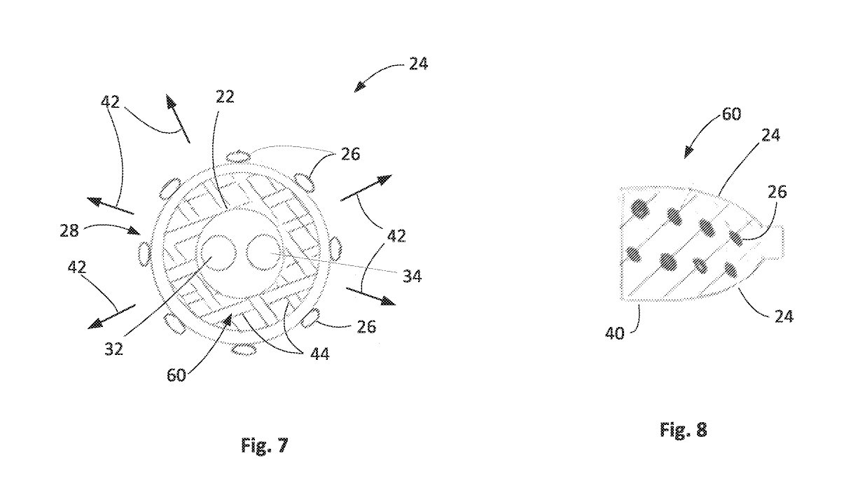

According to some embodiments, the expandable member may comprise a self-expanding mesh 40. An example of this is seen in the device 10 of FIGS. 1-4, for example. Mesh 40 may be similar to the mesh of a self-expanding stent, for example, and can have properties that permit the mesh 40 to reliably cycle through compression and expansion. For example, the mesh 40 may be comprised of nitinol, stainless steel or other appropriate material and may be heat treated such as through an annealing process. Within the lumen 38 of the guiding catheter 12, the mesh 40 can be in a compressed form, such that the mesh 40 can be exerting a radially outward force (as shown by the arrows 42 in FIG. 7). The radially outwards force can press the self-expanding mesh 40 against the inside surface of the guiding catheter 12. When exterior of the guiding catheter lumen 38, the mesh 40 can be allowed to expand, and depending on the degree of expansion that is permitted, the mesh 40 may or may not exert a radially outwards force. For example, the mesh 40 that is depicted in FIGS. 3 and 4 may be fully expanded, and thus not exerting a radially outwards force against a vessel wall.

The mesh used in various embodiments described herein may be sufficiently open to allow blood to flow at a normal rate or at a substantially normal rate and to cause little or no obstruction or substantially no obstruction of blood flow. For example, the mesh may be comprised of openings measuring between about 0.002 inches and about 0.4 inches. The mesh may be any size which is not too small to sufficiently impact blood flow, and not too large to lose sufficient strength to hold the vessel open in the event of spasm. In embodiments in which physiological changes are monitored, such as changes in blood flow, the mesh may be sufficiently open as to not interfere with such monitoring. For example, if the blood flow is slightly obstructed by the expanded mesh expandable member 24, such obstruction may not have an impact on the physiological monitoring because the monitoring detects a change in the physiological parameter caused by stimulation of a nerve in the vessel. Because the physiological parameter is monitored both before and after stimulation, with the expandable member 24 in an expanded state and in the same position in the vessel at both times, the impact of the expandable member 24 on the physiological parameter would exist at both times. Therefore, a change in the physiological parameter caused by stimulation should not be affected by the presence of the expandable member in the vessel, particularly if its impact on the physiological parameter is small.

In some embodiments, the expandable member may be a balloon. For example, the expandable member may include ablation elements on its surface and may be expanded by a clinician, such as by inflating the balloon with fluid through the catheter and into the expandable member.

In some embodiments, when compressed, the expandable member 24 such as the self-expanding mesh 40 can exert an even force about its perimeter, such that the force exerted on the inner wall of the guiding catheter 12 is substantially equal along the entire inner surface of the guiding catheter 12 that is in contact with the expandable member 24. In some embodiments, the expandable member 24 can be configured to remain co-axial with the therapeutic catheter 14, both when compressed within the guiding catheter 12, and when expanded as depicted in FIG. 3.

Other shapes of the expandable member 24 are also contemplated. FIGS. 7 and 8 depict a distal end view and a side view, respectively, of an alternative embodiment of the therapeutic catheter 14 of FIGS. 1-4. In FIGS. 7 and 8, the expandable member is a parabolic shaped expandable member 60, having an outer circumference that increases as the expandable member extends distally. In these Figures, the expandable member 60 is in an expanded form, which may be achieved by self-expansion after deployment out of the guiding catheter 12. The shape of parabolic shaped expandable member 60, being narrower at the proximal end, allows it to collapse back into an unexpanded shape when it is withdrawn into the guiding catheter 12 for repositioning or for removal at the end of a procedure. Electrodes 26 are located at various locations surrounding the expandable member 24. The expandable member 24 may be comprised of a self-expanding material such as a self-expanding mesh 40, to automatically expand from a compressed state within the guiding catheter 12 to an expanded state having a larger diameter upon deployment and with the largest diameter at the distal end of the expandable member 24.

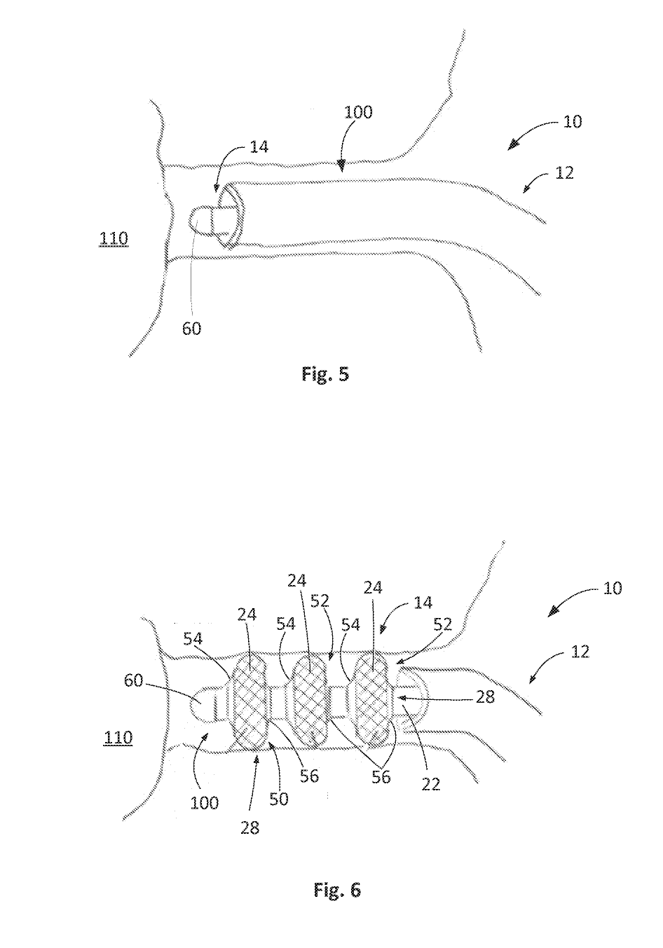

FIG. 5 shows a catheter 10 which can include a guiding catheter 12 and a therapeutic catheter 14, within the lumen of a renal artery 100 in proximity to the kidney 110 in accordance with some embodiments. FIG. 6 shows the therapeutic catheter 14 extended distally beyond the distal end of the guiding catheter 12. Additional details regarding the method of locating a guiding catheter 12 and a therapeutic catheter 14 within the lumen of an artery are discussed elsewhere herein.

In some embodiments, the expandable member 24 may comprise ablation elements and/or stimulation elements, such as one distally located ablation element 50 and two proximally located stimulation elements 52 as shown in FIG. 6. In the embodiment shown in FIG. 6, the expandable member 24 are comprised of self-expanding mesh and can have radially extending, somewhat cone-shaped, distal sidewalls 54, and/or radially extending, somewhat cone-shaped, proximal sidewalls 56. The distal and/or proximal sidewalls 54, 56 can attach the drum-shaped self-expanding mesh to the outer surface of the catheter portion 22 of the therapeutic catheter 14. In some embodiments, both the distal sidewall 54 and the proximal sidewall 56, or only one or the other, may be used to attach the drum-shaped self-expanding mesh to the catheter portion 22 of the therapeutic catheter 14. In the embodiment shown in FIG. 6, the therapeutic catheter also includes a diagnostic sensor 60 at its distal tip, which may be a blood velocity or pressure sensor, for example.

In some embodiments, a distal sidewall 54 and/or the proximal sidewall 56 may comprise an expanding mesh. In some embodiments, one or both sidewalls 54, 56 may comprise a surgical grade elastomer that can stretch and contract with sufficient flexibility to permit the expandable member 24 to reliably compress and expand.

In some embodiments, the proximal sidewall 56 may have a conical surface profile that is sufficiently shallow in shape that when the therapeutic catheter 14 is moved proximally relative to the guiding catheter 12, the proximal sidewall 56 contacts the distal end 18 of the guiding catheter 12. As the therapeutic catheter 14 continues to move proximally relative to the guiding catheter 12, the continued contact between the proximal cone-shaped sidewall 56 and the distal end 18 of the guiding catheter 12, causes the self-expanding mesh to contract in diameter, thereby easing the introduction of the expandable member 24 into the lumen 38 of the guiding catheter 12. Alternatively, other mechanisms may be used to allow the expandable member 24 to contract after deployment. For example, in some embodiments, a drawstring which may be a wire can be threaded through each expandable member 24 such that pulling on the drawstring can cause the member 24 to contract. Releasing the drawstring can then permit the member 24 to self-expand.

In some embodiments, the expandable member may be reduced in size to fit within the guiding catheter 12 by retracting the therapeutic catheter into the guiding catheter 12.

In some embodiments, the guiding catheter 12 can have a non-stick coating (for example, polytetrafluoroethylene, more commonly known as TEFLON) applied to the inner surface of its lumen 38. This can then permit the therapeutic catheter 14 to be more easily deployed from within the guiding catheter. Moving a compressed self-expanding mesh 40 out of a guiding catheter 12, that has a non-stick surface, can result in a deformation-free expanded member 24, such as a deform free expanded mesh 40.

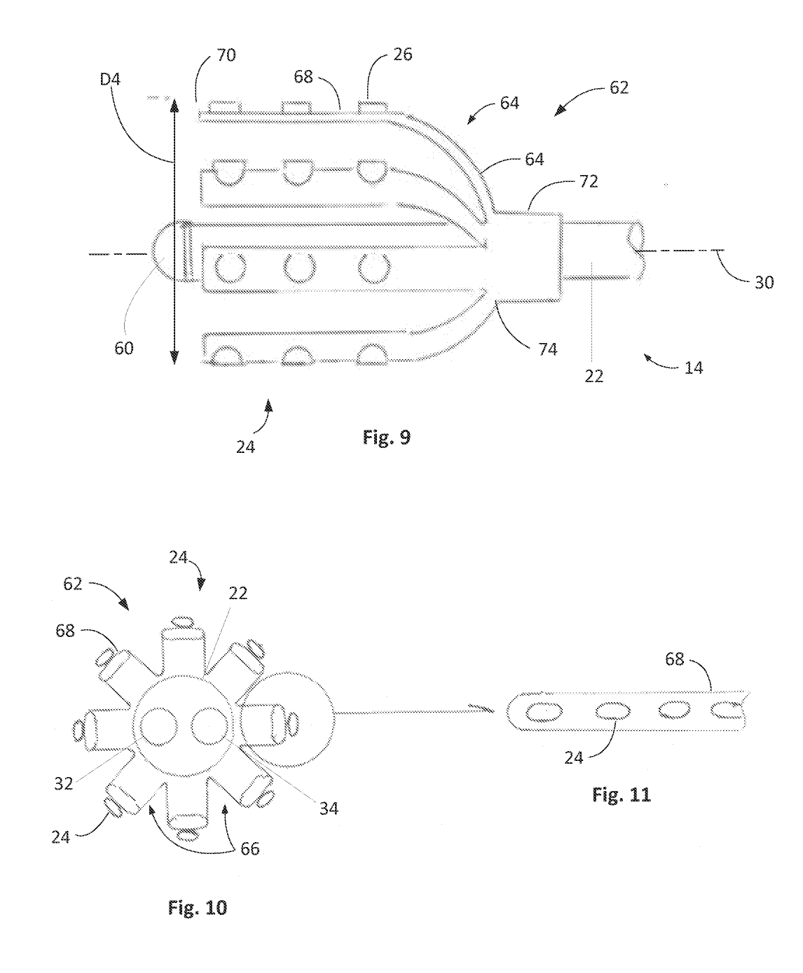

According to some embodiments, the expandable member 24 may comprise a basket shaped expandable member 62 having one or more arms 64, such as the plurality of arms 64 as shown in the embodiment depicted in FIGS. 9-11. The arms 64 may extend radially outward as well as distally along the length of the catheter portion 22 of therapeutic catheter 14 to encircle the catheter portion 22, taking on a basket-like shape. In the embodiment shown, the proximal portion 66 of the arm 64 extends radially outward and also somewhat distally, while the distal portion 68 extends distally, parallel with the catheter portion 22. A side elevational view is shown in FIG. 9 while a distal end view is shown in FIG. 10. The distal portion 68 may be longitudinal members that extend distally substantially parallel with the central axis 30 of a therapeutic catheter 14. This can give the basket-shaped expandable member 62 a diameter of D4 when expanded, as depicted in FIG. 9. The distal portion 68 of the arms 64 can have a free end 70 and an end that is joined with the proximal portion 66. Each of the arms 64 can be joined to a central collar 72 at a juncture 74 encircling the catheter portion 22. In some embodiments the collar 72 and the arms 64 can be integrally formed, such that the juncture 74 is a feature of the integrally formed part. According to some embodiments, the distal portion 68 can have multiple electrodes 26 along its length on its outer surface (as shown in FIG. 9-11). In some embodiments, the electrodes 26 can be screen printed onto the arm material directly. In some embodiments, the arms 64 can be inflatable structures that expand to the general shapes shown in FIGS. 9 and 10 once they are inflated by a clinician. In some embodiments, the juncture 74 can be a biased hinge. In some embodiments, the proximal portion 66 can be relatively more flexible than the distal portion 68.

In some embodiments, the features of the basket-shaped expandable member 62 permit it to contract to an overall diameter that is less than D2, such that it can fit within the lumen of guiding catheter 12. In some embodiments, the flexibility of the proximal portion 66 and/or the juncture 74 can permit the arms 64 to remain substantially parallel with the axis 30 of the therapeutic catheter 12 throughout their lengths when the basket-shaped expandable member 64 is contracted from a fully expanded state to a fully contracted state, such that throughout the majority of the contraction motion, the arms 64 can remain substantially parallel with the axis 30. In some embodiments that use inflatable arms 64, the arms 64 can be at least partially deflated to allow for the therapeutic catheter 12 to fit within the lumen of a guiding catheter 12 and/or to ease repositioning of the therapeutic catheter 14 within an artery.

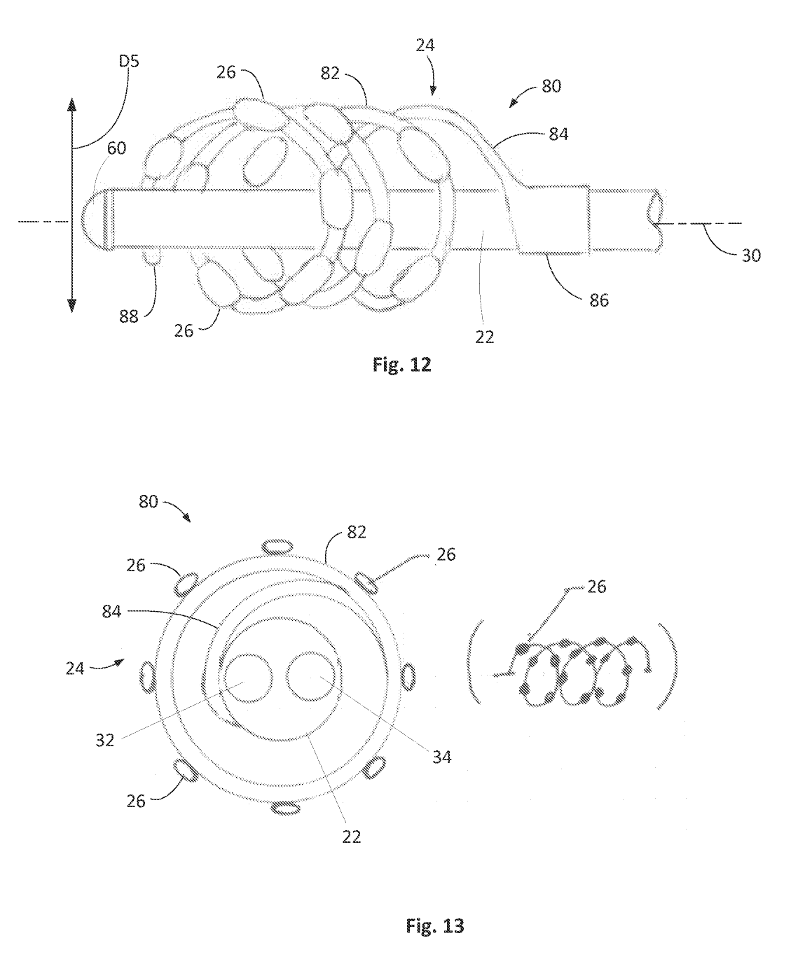

In some embodiments, the expandable member 24 may have the shape of a spring coil form 80 as shown in FIGS. 12-13, for example. The spring coil form 80 may include a first coil portion 82, a second coil portion 84 and a collar portion 86, as depicted in FIG. 12 that shows the spring coil form 80 in a side elevation view, and in FIG. 13 that shows the spring coil form 80 in a distal end view. The first coil portion 82 may have the general shape of a spring: a curve that is defined by a point moving around, and simultaneously advancing along the axis 30 of the therapeutic catheter 14 at a uniform distance from the axis 30. The first coil portion 82 can have multiple electrodes 26 along its length on its outer surface. In some embodiments, the first coil portion 82 may have only a single electrode 26 on its outer surface. In its expanded state, the first coil portion 82 can have a diameter of D5, and can have a free end 88, and an end that is joined to the second coil portion 84. The second coil portion 84 can connect the first coil portion 82 to the collar portion 86. The second coil portion 84 can be generally helically shaped, having a contour that can be defined by a point moving around axis 30, and simultaneously advancing along, and steadily increasing (or diminishing) its distance from axis 30 of the therapeutic catheter 14. The collar portion 86 can join the second coil portion 84 to the catheter portion 22 of the therapeutic catheter 14.

In some embodiments, the materials, combined with the physical structure, of the spring coil form 80 permit it to contract to an overall diameter that is less than D2, such that the spring coil 80 can fit within the lumen of guiding catheter 12. In some embodiments, the flexibility of the first coil portion 82 and the second coil portion 84 permit the spring coil form 80 to contract in diameter. The spring coil may be a metal coil or a shaped polymer such material such as PEBAX or polyurethane, for example.

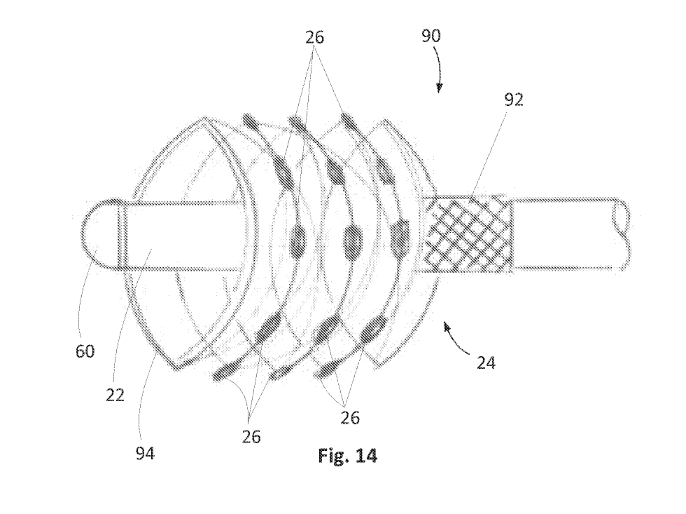

A further alternative embodiment of a therapeutic catheter 14 including a plurality of expandable member 24 and electrodes 26 is shown in FIG. 14. In this embodiment, the expandable member 24 include a proximal collar 92 surrounding the catheter portion 22 and a plurality of concave disk shaped expandable member 94. In their expanded form as shown in FIG. 14, members 94 are adjoined to the collar 94 at their proximal ends, and flare radially outward as they expand distally. A plurality of electrodes 26 encircle the outer perimeter of the concave disk shaped expandable member 92 along their distal edges, though alternative locations may also be used. As in the other expandable member, the concave disk shaped expandable member 94 may be comprised of an expandable mesh that may be self-expanding. The shape of the concave disk shaped expandable member 94 assist the members 94 in collapsing, beginning at their proximal ends, as the therapeutic catheter 14 is retracted into the guiding catheter 12.

Some of the embodiments of the devices described herein may include expandable member 24 that can self-expand. These elements 24 may be configured and sized such that they will fit within the lumen of a guiding catheter 12 when contracted. When expanded, these elements 24 can abut the inner walls of an artery, such that they apply an outwards radial force on the inner walls of the artery. These elements 24 may be further configured and sized such that the magnitude of the radial force is sufficient to hold open the artery during an arterial spasm.

In addition, some of the embodiments of the expandable member 24 may permit blood flow when fully expanded within an artery. The expandable member may be sized and constructed as to have a marginal impact, such as little or no impact, on the blood flow through, and the blood pressure within, the artery. For example, the expandable member 24 may comprise a mesh or other wire formation to have minimal impact on blood flow.

As described elsewhere herein, electrodes 26 on the expandable member 24 may be used to locate and map nerves such as sympathetic nerves, to stimulate and/or monitor the nerves, and/or for ablating the nerves. In some embodiments, the expandable member 24 may be comprised of a self-expanding mesh and can have individual metal strands that are coated with an insulating layer (e.g. a coating of silicone). These coated strands can be incorporated within the wire matrix itself that forms the mesh. In this manner, some or all of these coated metal strands can terminate at an electrode 26, such that each such connected electrode 26 can be individually pulsed for either ablation, or for stimulation or mapping. In some embodiments, each electrode 26 can be individually electrically connected by a conductor (e.g. a wire) to a power generator and a controller.

In some embodiments, some or all of the electrodes 26 used for mapping, stimulating, monitoring and/or for ablation may be traditional conductive metal electrodes, for example. In some embodiments, these electrodes 26 may also be used for temperature monitoring during ablation, for sensing electrical conduction to monitor the progress of ablation, or for other purposes. Any type of electrode suitable for the purposes described herein may be used.

In some embodiments, the electrodes 26 supply electrical energy, while in other embodiments they may supply alternative forms of ablative energy. Alternatively, in any of the embodiments described herein, a different stimulation or ablation element may be used in place of the electrode 26. In addition, the expandable member themselves may be comprised of a conductive mesh and may themselves act as electrodes or they may be insulated by a coating such as TEFLON, polyurethane, polyimide, or other coating.

An example of a process for mapping and ablating a nerve within the wall of an artery that includes the application of a radial force will now be described. In order to describe the methods with clarity, specific reference will be made to renal nerve ablation. It will be appreciated, however, that embodiments of the methods disclosed herein can be applicable to treat or alter other sympathetic nerve functions and at other locations and may be used in other ablation applications. Accordingly, references to particular mapping and ablation locations should not be read to limit applicability, but rather as clarifying examples.

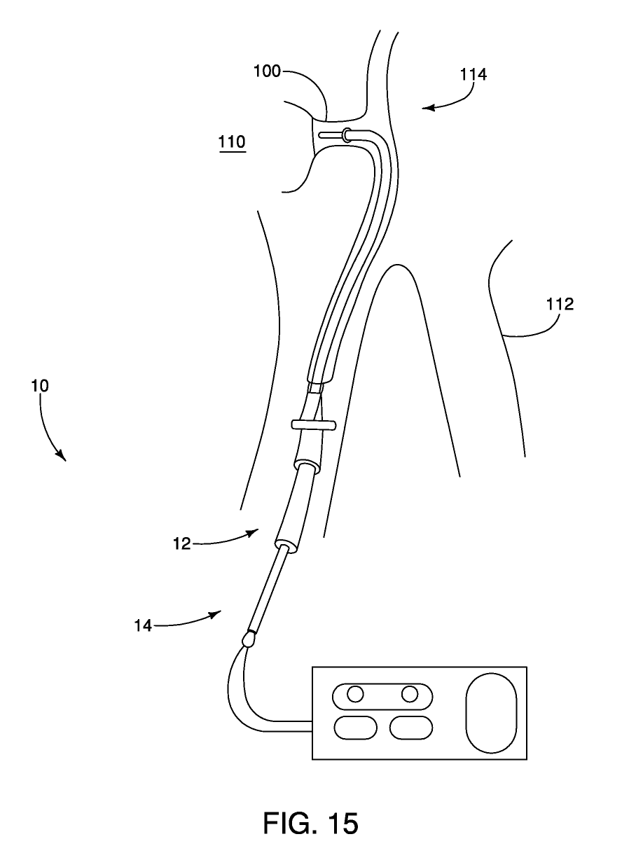

Referring now to FIG. 15, a catheter 10 may be percutaneously introduced into a patient 112 and advanced through the arterial system 114 into the lumen of the renal artery 100 or other artery or blood vessel. Normal blood flow in the renal artery 100 may be measured to determine a baseline. In some embodiments, a guiding catheter 12 can be used to guide a therapeutic catheter 14. If such a guiding catheter 12 has been used, it can be proximally retracted in order to reveal the therapeutic catheter 14 to the renal artery 100, or the guiding catheter 12 may be held stationary as the therapeutic catheter 14 is advanced distally out of it. According to some embodiments, the device may be sized and/or the size may be selected by the clinician for use at the treatment location within an artery such that the configuration of the therapeutic catheter 14, once removed from within the lumen of the guiding catheter 12, can cause a radial force to be applied to the inside walls of the artery 100. Such a force can be due to the radially outwards expansion of one or more expandable member 24 of the therapeutic catheter 14. Such a force can be of sufficient magnitude to hold open the artery 100 in the event that an arterial spasm were to occur.

Electrodes 26 on the perimeter of the expandable member 24 may be brought into proximity or contact with the artery wall due to the radially outwards expansion of the elements. Stimulation may optimally be applied by a first group of one or more electrodes 26 at a first location or locations and detection of stimulated nerve activity may be monitored at a second location. A second group of one or more electrodes 26 can then be chosen and this process may optimally be repeated at a third location, etc. until the stimulation is detected by one or more of the other electrodes at a fourth location, etc., or until a physiological response is detected. This can be an indication that electrical conduction has occurred through a nerve branch or branches and that a nerve branch or branches have, therefore, been located as a monitoring and/or treatment location. In some embodiments, the therapeutic catheter can be rotated about its own axis prior to selecting a second group of one or more electrodes 26 for stimulation. Alternatively, the position of the therapeutic catheter 14 may be maintained, and the positioning of the multiple electrodes around the perimeter of the therapeutic catheter 14 may be used to test or monitor the artery in various locations without repositioning. For example, a square wave stimulation pulse may be delivered having an amplitude of about 40 volts, a width of 3 milliseconds, and a frequency of 5 cycles/second. A reduction in blood flow of about 25 to 100% may occur. The change in blood flow may be measured during nerve stimulation, immediately after nerve stimulation, or after some time delay. The measurement of a baseline blood flow and a blood flow during or after stimulation of the nerve may be repeated one or more times, such as after a delay or rest period, for verification.

Ablation of the nerve (after optimally identifying the location as described above and monitoring a response to stimulation) may then be performed, using the same group of one or more electrodes 26 as used for successful mapping and/or stimulation, or using a separate electrode or method, with or without moving the therapeutic catheter 14 from the location at which the successful mapping and/or stimulation were performed. Following ablation, the same stimulation process as performed before ablation may optionally be repeated by delivering an identical stimulation pulse, and the blood flow or nerve conduction may be measured again in the same manner in which the baseline measurement was obtained before ablation. (A new baseline physiological measurement may be obtained first, after ablation but prior to stimulation of the nerve, or the original baseline measurement may be used.) The difference in the change in blood flow caused by stimulation of the nerve, or the change in nerve conduction, before and after ablation may then be calculated. If the difference correlates to adequate ablation, the process may be stopped. However, if the difference is not sufficient/too small, the steps of ablating and measuring blood flow or nerve conduction, such as without moving the therapeutic catheter, before, during and/or after stimulation may be repeated until the difference in change in blood flow, and/or the change in nerve conduction, is sufficient to indicate the desired amount of denervation has been achieved.

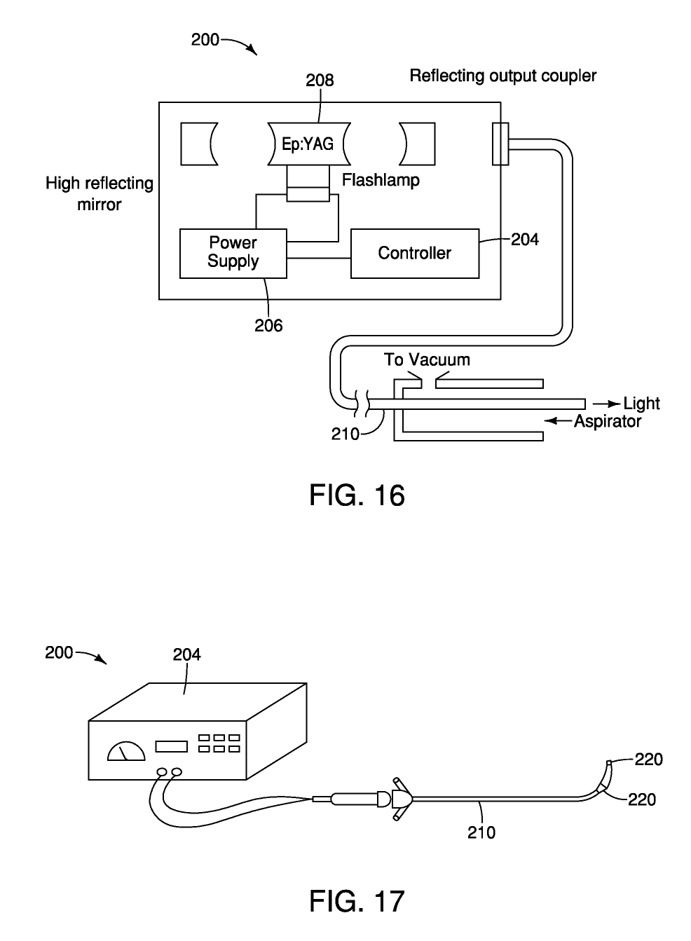

An example of a nerve treatment location mapping, monitoring, and ablation system is shown in FIG. 16. In this embodiment, the system delivers laser energy for ablation, but other forms of ablative energy could alternatively be used. The system 200 includes a controller 204 coupled to a power supply 206. A laser energy source 208, which in this example is an Erbium doped solid state gain medium which may have first and/or second resonant cavities and first and/or second couplers. The system further includes a catheter 210 for treatment delivery. A second example of a nerve mapping and ablation system 200 is shown in FIG. 17, which depicts a controller 204 connected to a steerable ablation catheter 210. Electrodes 220 are shown on the distal end of the catheter 210. In some embodiments of this catheter 210 as well as the others described herein, the catheter may also include one or more sensors for measuring one or more physiological parameters within the renal artery or other artery or vessel.

In some embodiments, some or all of the electrodes may be printed screen electrodes on a surface, such as on a catheter or expandable balloon. Such printed screen electrodes may include a printed electrode of a conductive material such as a conductive ink such as a platinum ink and printed conductors on a flexible film such as a polyimide film. The printed electrodes including the film may be applied directly to the surface and the printed conductors may attach proximally to a conductive wire. The printed electrodes can provide multiple data collection points to increase diagnostic and therapeutic capabilities and can reduce assembly complications while maintaining catheter flexibility, without increasing the catheter diameter.

The description provided herein is exemplary in nature and is not intended to limit the scope, applicability, or configuration of the invention in any way. Rather, the description provides practical illustrations for implementing various exemplary embodiments. Examples of constructions, materials, dimensions, and manufacturing processes are provided for selected elements, and all other elements employ that which is known to those of skill in the field. Those skilled in the art will recognize that many of the examples provided have suitable alternatives that can be utilized.

What is claimed is:

1. A method of ablating nerves within an artery of a patient at a treatment location comprising:
selecting an ablation device comprising an outer guiding catheter and an inner treatment catheter, the treatment catheter comprising an expandable element and an ablative element located on the expandable element, wherein the selection of the ablation device is such that the expandable element is of a size to apply an outward force against a portion of an inner wall of the artery substantially sufficient to hold the artery open during an arterial spasm event;
advancing the ablation device to the treatment location;
positioning the treatment catheter out of the guiding catheter;
expanding the expandable element, wherein after expansion, the expandable element applies the outward force against the portion of the inner wall of the artery; and
after expanding the expandable element and without moving the treatment catheter, ablating the inner wall of the artery using the ablation element.

2. The method of claim 1 wherein the ablation device is selected such that the expandable element abuts the inner wall of the artery around a circumference of the artery.

3. The method of claim 1 wherein the treatment catheter comprises a plurality of ablative elements on an outer surface of the expandable element positioned to abut the inner wall of the artery around the circumference of the artery.

4. The method of claim 3 further comprising ablating with one or more first ablative elements of the plurality of ablative elements and then ablating with one or more second ablative elements of the plurality of ablative elements without moving the expandable treatment catheter.

5. The method of claim 1 further comprising emitting a stimulation pulse prior to ablation and detecting a physiological response using the ablation device.

6. The method of claim 5 further comprising emitting a second stimulation pulse after ablation and detecting the physiological response using the ablation device.

7. The method of claim 6 wherein the physiological response comprises blood flow.

8. The method of claim 7 wherein the treatment location is the renal artery.

9. A method of monitoring nerve activity of a patient within an artery comprising:
selecting a monitoring device comprising an outer guiding catheter and an inner monitoring device, the monitoring device comprising an expandable element, an electrical stimulation element and an electrical detection element, wherein each of the electrical stimulation element and the electrical detection element are located on an outer surface of the expandable element, and wherein the expandable element is configured to apply an outward force against a portion of an inner wall of the artery substantially sufficient to hold the artery open during an arterial spasm event;
advancing the ablation device to the treatment location;
advancing the monitoring device out of the guiding catheter;
expanding the expandable element, wherein after expansion, the expandable element applies the outward force against the portion of the inner wall of the artery;
emitting a first stimulation pulse from the stimulation element; and
detecting nerve activity produced in response to the first stimulation pulse with the detection element.

10. The method of claim 1 wherein the monitoring device further comprises an ablation element located on the expandable element, further comprising:
after detecting nerve activity produced in response to the first stimulation pulse, ablating the inner wall of the artery using the ablation element;
after ablating, emitting a second stimulation pulse from the stimulation element; and
detecting the presence or absence of nerve activity produced in response to the second stimulation pulse with the detection element.

11. The method of claim 10, wherein the monitoring device is selected such that the expandable element abuts the inner wall of the artery around a circumference of the artery.

12. The method of claim 11 wherein the artery comprises a renal artery.

13. An ablation device comprising:
a guiding catheter having an inner lumen;
a treatment catheter within the guiding catheter, the treatment catheter comprising:
a plurality of electrodes;
an expandable element having a first state when located within the guidance catheter lumen having a first diameter, and a second state that is expanded relative to the first state and having a second diameter and an outer surface, wherein the second diameter is greater than the first diameter, and wherein size that presses against the inner surface of a second lumen having a second diameter, the second diameter greater than the first diameter; wherein the plurality of electrodes are located on the outer surface of the expandable element,
wherein when the expandable element is expanded in an artery having a diameter with less than the second diameter, the expandable element is configured to apply a radially outward force against the artery sufficient to hold the artery open during a spasm event, and
wherein the expandable element is configured to expand from the first state to the second state for ablation, and then to return to the first state to allow the treatment catheter to be retracted into the guiding catheter for removal of the ablation device.

14. The ablation device of claim 13 wherein the expandable element is self-expanding.

15. The ablation device of claim 14 wherein the expandable element comprises a mesh.

16. The ablation device of claim 15 wherein the expandable element is cylindrical.

17. The ablation device of claim 16 wherein the expandable element has parabolic shaped sidewalls.

18. The ablation device of claim 13 wherein the expandable element is basket shaped and comprises a plurality of arms.

19. The ablation device of claim 18 wherein the arms are inflatable.

20. The ablation device of claim 13 wherein the expandable element is a spring coil.
